(19) United States
(12) Patent Application Publication
(10) Pub. No.: US 2006/0263762 A1
(43) Pub. Date: Nov. 23, 2006

(54) CYTOSKELETAL INTEGRITY TESTING AS A FILTER FOR CREATING HOMOGENEOUS SAMPLES FOR FURTHER ANALYSIS

(76) Inventor: Christian Walker, Worcester, MA (US)

Correspondence Address: BLODGETT BLODGETT, 43 HIGHLAND STREET, WORCESTER, MA, 016092797, US

(21) Appl. No.: 11/383,425

(22) Filed: May 15, 2006

Related U.S. Application Data
(60) Provisional application No. 60/594,897, filed on May 16, 2005.

(51) Int. Cl. C12N 5/06; C12Q 1/00

(52) U.S. Cl. 435/4; 435/325

(57) ABSTRACT

The present invention is generally directed to the integration of cytoskeletal manipulation with genetic and proteomic analysis. “Cytoskeletal manipulation” references any technology that uses either cytoskeletal integrity or cell adhesion to isolate symptomatic cells from asymptomatic cells as a gatekeeping step for genetic and proteomic analysis. As used in the present invention, the term “isolate” means separating symptomatic from asymptomatic cells based on the cells' demonstration of the relevant associated intracellular or intercellular architectural properties. The further analysis of cells sorted using this invention may include any advanced form of chemical, physical, or biological testing including all forms of genetic and protein analysis, or alternative form of biological expression analysis that is now known or might in the future be developed.

### CROSS-REFERENCE TO RELATED APPLICATIONS

This application claims the benefit under 35 U.S.C. section 119(e) of U.S. Provisional patent application No. 60/594897 filed May 16, 2005, all of which is hereby incorporated by reference.

### STATEMENT REGARDING FEDERALLY SPONSORED RESEARCH OR DEVELOPMENT

This invention has been created without the sponsorship or funding of any federally sponsored research or development program.

### REFERENCE TO SEQUENCE LISTING, A TABLE, OR A COMPUTER PROGRAM LISTING COMPACT DISK APPENDIX

Not applicable.

### BACKGROUND OF THE INVENTION

This invention relates to the analysis of groups of biological cells and particularly groups of biological cells made up of a mixture of cells having investigational interest and cells not having investigational interest.

In a typical case, cancer results from a single localized incident of genetic change, adulteration, or damage. The damage occurs within a stretch of DNA that codes for protein. A variety of specific events of damage to DNA coding for a variety of proteins have been documented. These lead to cancers with widely varying causes, pathologies, and prognoses. The cancers based on these different mechanisms and manifested in different tissues respond differently to various therapeutic strategies. It is within this context that the medical research community is working to develop rapid screening techniques capable of identifying one cause out of thousands of possibilities for a given cancer.

One of the secondary indicators known to be a diagnostic of neoplastic status (“cancer”) in cells is cytoskeletal integrity. The likely source of decreased cytoskeletal integrity is in an altered proteinaceous composition of the cytoskeleton, specifically an alteration of the relative proportions of interacting proteins in quaternary structural complexes. Obscuring the apparent simplicity of this picture, however, is the fact that a number of protein classes play various structural roles in the cytoskeletal architecture of any cell, including intermediate filaments, keratins, actins, myosins, microtubule associated proteins (MAPs), Tau proteins, spectrins, villin, synaptophysin, desmosomal proteins, and even cell adhesion molecules. The relative proportions of these protein types and of the quaternary interactions between the proteins vary substantially between various differentiated tissues. Accordingly the scientific community is still a long way from a comprehensive understanding of the manner in which the concerted action of these proteins determines the overall cytoskeletal properties of a single cell, let alone a fully-formed insight into the cascade of regulatory interactions that cause cancer cells to display their various forms of changed cytoskeletal integrity. Research has been directed to using optical methods to test the cytoskeletal integrity of cells.

It has been more than three decades since radiation pressure from lasers was first used to physically manipulate microscopic particles by a crude “pushing” attributable to simple momentum transfer. Two trends have characterized the intervening period. Improved control has permitted the manipulation of ever-smaller particles, and the use of multiple lasers configured in appropriate geometries has permitted more sophisticated trapping and manipulation of these particles. Dielectric materials, including cell membranes, display two well-characterized responses to such light sources, both of which are exploited in various micro-manipulation tools. The first is the classic and universal response of all matter, dielectric or otherwise, to incident radiation that is absorbed and back-scattered: momentum-transfer from the incident photons results in a physical force in the direction of the original path of the incident photons. The second is unique to dielectric materials and is a force directed up-gradient towards the highest density of photons, typically an optically manipulated focal point. While most micro-manipulation tools exploit the latter, the optical stretcher exploits the former.

In an optical stretcher, the output of two coherent light sources or the light of a single source split into two light paths is aligned coaxially in opposed directions. Though the initial expectation was that the opposed streams of radiation pressure would compress the cell, it was instead discovered that each light beam had the strongest repulsive force on the distal rather proximal cytoplasmic membrane surface. Common speculations include that this is attributable either to the differing dielectric properties of the interior and exterior of the cell or to the geometry of a spreading beam. The cytoarchitectural properties of cells such as cytoskeletal integrity and cell adhesion can be diagnostic indicators of neoplasticity (cancerous behavior). Knowledge of the underlying origins of the changes in cytoskeletal integrity is less important to the diagnostic value than the simple measurable correlations between cancer status and the associated change in the modulus of linear elongation. Research has been done to demonstrate the preliminary viability of differentiating cancer and non-cancer cells based on differences in cytoskeletal integrity using the optical stretcher. False positives and false negatives, however, contaminate the sample being isolated hampering further analysis.

This proposed method provides an effective method for identifying that some members of a group of cells display cytoarchitectural properties different from cytoarchitectural properties of the other members of the group and the presence of a heterogeneous population makes it very difficult to analyze the atypical cells of interest while they are present in a mixed group.

These and other difficulties experienced with the prior art processes have been obviated in a novel manner by the present invention.

It is, therefore, an outstanding object of the present invention to provide a system for identifying cells having atypical cytoarchitectural properties in a mixture of cells with and without the atypical cytoarchitectural properties, and separating the cells having atypical cytoarchitectural properties from the mixture.

It is a further object of the invention to provide a system for identifying cells having atypical cytoarchitectural properties in a mixture of cells with and without the atypical cytoarchitectural properties, and separating the cells having atypical cytoarchitectural properties from the mixture, which system is capable of being able to create homogenous population of cells based upon cytoskeletal integrity as a selectable bio-marker, which can be operated with high accuracy at a low cost, and which is capable of providing a long and useful life with a minimum of maintenance.

With these and other objects in view, as will be apparent to those skilled in the art, the invention resides in the combination of parts and steps set forth in the specification and covered by the claims appended hereto, it being understood that changes in the precise embodiment of the invention herein disclosed may be made within the scope of what is claimed without departing from the spirit of the invention.

### BRIEF SUMMARY OF THE INVENTION

The present invention is generally directed to the integration of cytoskeletal manipulation with genetic and proteomic analysis. “Cytoskeletal manipulation” references any technology that uses either cytoskeletal integrity or cell adhesion to isolate symptomatic cells from asymptomatic cells as a gatekeeping step for further analysis. As used in the present invention, the term “isolate” means separating symptomatic from asymptomatic cells based on the cells'demonstration of the relevant associated intracellular or intercellular architectural properties. The further analysis of cells sorted using this invention may include any advanced form of chemical, physical, or biological testing including all forms of genetic and protein analysis, or alternative form of biological expression analysis that is now known or might in the future be developed.

### BRIEF DESCRIPTION OF THE DRAWING

The character of the invention, however, may best be understood by reference to one of its structural forms, as illustrated by the accompanying drawings, in which:

FIG. 1 is a flowchart diagram of steps of a process used for cytoskeletal integrity testing as a gate keeper for creating homogenous samples for further analysis.

### DETAILED DESCRIPTION OF THE INVENTION

The present invention is generally directed to the integration of cytoskeletal manipulation with cell (such as genetic and/or proteomic) analysis. “Cytoskeletal manipulation” references any technology that uses either cytoskeletal integrity or cell adhesion to isolate symptomatic cells from asymptomatic cells as a gatekeeping step for further analysis. As used in the present invention, the term “isolate” means separating symptomatic from asymptomatic cells based on the cells'demonstration of the relevant associated intracellular or intercellular architectural properties. The genetic and proteomic analysis technology may include any advanced form of genetic and protein analysis or expression analysis susceptible to automation that is now known or might in the future be developed.

For the purposes of the present invention, the term “symptomatic cells” refers to cells that display the cytoarchitectural feature associated with the sub-population of cells that is being isolated for transfer to genetic, or protein analysis or expression analysis. The term is not intended to imply that the symptomatic cells are necessarily demonstrating pathological behavior, as discussed below for in-vitro gene therapy applications. Thus, it is important to recognize that the intended connotation of the terms “symptomatic” and “asymptomatic” is commensurately broad.

In one embodiment, the integrated technologies may be used in monitoring gene therapy. In particular, the therapeutic progress of gene therapies currently under federal scrutiny may be tracked. In this case, the introduced and dispersed cells typically contain different genetic information than their neighbors. In another embodiment, modified cells may be sorted from unmodified cells in vitro during the preparation of a population of modified cells for introduction into a patient during gene therapy. In another embodiment, the isolation of hematopoietic stem cells may be sorted from blood. In another embodiment, the effect of protein based drugs may be scrutinized by tracking changes in cytoskeletal integrity and effect of protein-protein interactions monitored.

In all these applications, cells that are characteristic of a patient's native genetic information and protein expression patterns are labeled asymptomatic, and those with the new genetic information and altered protein expression patterns are labeled symptomatic. It is noted that these labels are assigned while acknowledging that it may be the modified cells that behave normally while the patient's native genetic code and protein expression patterns that produces pathological symptoms. This applies whether the selection criteria is functional, secondary, or purely a marker introduced to facilitate identification of modified cells. Other applications include gatekeeping technology to isolate symptomatic cells from asymptomatic cells as a prerequisite to unambiguous genetic, or protein analysis or expression analysis, regardless of whether the mechanism is based on cytoskeletal integrity, cell adhesion, or other native or introduced characteristics.

It may be desirable to link the various techniques (i.e., cytoskeletal manipulation, genomic and proteomic analysis) in such a way that symptomatic cells could be taken directly from the cytoskeletal manipulation device to the genetic and protein analysis devices with an intervening step involving established techniques for lysing the selected cells. This intervening step may include, but is not limited to, extracting the DNA and/or RNA, digesting the DNA (and RNA if appropriate), and amplifying the DNA and isolating proteins from cell lysate for further testing. It may be desirable to link the various techniques (i.e., cytoskeletal manipulation, genomic and proteomic analysis) in such a way that symptomatic cells could be taken directly from the cytoskeletal manipulation device to the genetic and protein analysis devices with an intervening step involving established techniques for lysing the selected cells. This intervening step may include, but is not limited to, extracting the DNA and/or RNA, digesting the DNA (and RNA if appropriate), and amplifying the DNA and isolating proteins from cell lysate for further testing.

One embodiment of the present invention includes a fully automated device that incorporates the preceding capabilities and advantageously simplifies cancer diagnosis and offers widespread acceptance and use of cytoskeletal dynamics as a cellular marker and potential diagnostic tool based on the availability of homogenous samples. For example, a technician seeing this invention only as an increased convenience might use a cytoskeletal manipulation device then manually transfer the sample for genetic, or protein analysis, missing an important application. An important role that is being filled by this embodiment of the present invention is the gatekeeping role of the cytoskeletal manipulation in eliminating asymptomatic cells from the genetic, or protein analysis in order to create a homogenous sample, overcoming the most difficult factors in genetic and protein analysis.

Further, the present invention provides for the use of an optical stretcher to test cytoskeletal integrity as more than a screening technique for cancer. Specifically, in some embodiments, this use of an optical stretcher may be used for the subsequent transfer of cells having a putative cancer status into a device to determine the molecular biological origins of the pathological behavior. An advantage to this is improved diagnostic certainty resulting from the introduction of only confirmed tumor cells into the genetic and protein analysis. The ability to use a device not merely as a diagnostic tool to stretch cells, but also as a sorting device to select a homogeneous sample of putative cancer cells from a tissue sample containing a mixed population of cells, provides a basis for screening cancers at a genetic and protein level. The individualized ability to screen cancers at the genetic and protein level allows physicians to choose a treatment regimen targeted to the genetic origins and protein markers of an individual's cancer. Another advantage is that it lowers cost by reserving the expensive use of microarrays for tissue samples of putative cancer cells. The phrase “homogenous sample of putative cancer cells,” implies that each cell demonstrating a greater degree of stretchability is a daughter cell of a single original cell that experienced a cancer-initiating event.

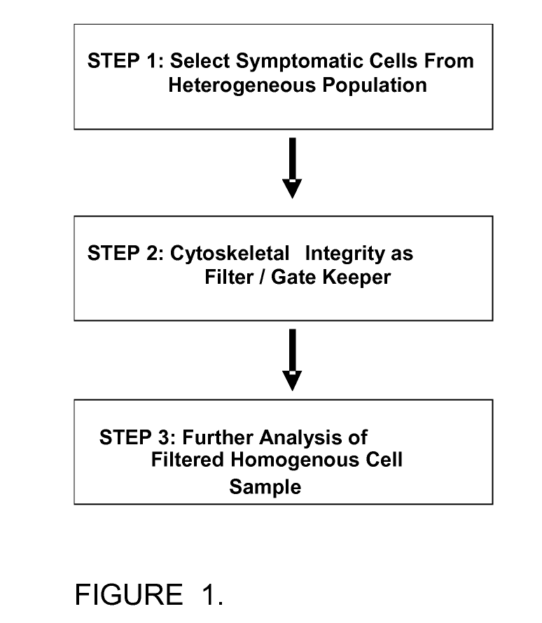

FIG. 1 is a flowchart diagram of steps in a process used for cytoskeletal integrity testing and subsequent genetic and protein analysis. In Step 1, selecting symptomatic cells from a population of suspected cells may employ any test for cytoskeletal integrity. In Step 2, separating symptomatic cells from a population of suspected cancer cells may be accomplished by removing asymptomatic cells, leaving only symptomatic cells in the sample, or removing symptomatic cells into a homogeneous sample. Preparing a homogeneous sample for further testing may include any known method of lysing the cell, removing the cells genetic material and proteins. In Step 3, testing the sample using genetic and protein analysis may include methods of DNA, RNA and protein analysis.

The invention includes use of the technology for cell sorting applications for: 1. separation of rare cell populations (stem cell and dendritic cells), 2. gene therapy, 3. reproductive services, 4. diagnosis of disease (cancer, autoimmune and viral infections), and/or 5. to create populations of cells that can monitor drug, or therapeutic intervention against disease 6. To serve as a biomarker for positive, or negative selection in any population of cells where cytoskeletal integrity is an inherent selectable marker of biological importance.

While it will be apparent that the illustrated embodiments of the invention herein disclosed are calculated adequately to fulfill the object and advantages primarily stated, it is to be understood that the invention is susceptible to variation, modification, and change within the spirit and scope of the subjoined claims. It is obvious that minor changes may be made in the form and construction of the invention without departing from the material spirit thereof. It is not, however, desired to confine the invention to the exact form herein shown and described, but it is desired to include all such as properly come within the scope claimed.

The invention having been thus described, what is claimed as new and desire to secure by Letters Patent is:

What I claim as my invention is:

1. A method for creating homogeneous cell samples, comprising the steps of: testing a population of suspect cells;
selecting at least one symptomatic cell from said population; and
transferring said at least one symptomatic cell from said population into a homogeneous sample.

2. A system for filtering cells, comprising:
means for testing a population of cells;
means for separating symptomatic cells from asymptomatic cells into a homogeneous sample; and
means for transferring said symptomatic cell homogeneous sample.
